(19) United States
(12) Patent Application Publication
(10) Pub. No.: US 2024/0198423 A1
(43) Pub. Date: Jun. 20, 2024

(54) MONOCRYSTALLINE GOLD MICROPLATES METHODS OF FABRICATION THEREOF AND DEVICES COMPRISING SAME

(71) Applicant: B. G. NEGEV TECHNOLOGIES AND APPLICATIONS LTD., AT BEN-GURION UNIVERSITY, Beer Sheva (IL)

(72) Inventor: Muhammad Y. BASHOUTI, Majd al-Krum (IL)

(73) Assignee: B. G. NEGEV TECHNOLOGIES AND APPLICATIONS LTD., AT BEN-GURION UNIVERSITY, Beer Sheva (IL)

(21) Appl. No.: 18/287,108

(22) PCT Filed: Apr. 19, 2022
(86) PCT No.: PCT/IL2022/050409

§ 371 Date: Oct. 16, 2023

Related U.S. Application Data
(60) Provisional application No. 63/176,407, filed on Apr. 19, 2021.

(51) Int. Cl. B22F 9/24; B22F 7/08; G01N 27/327

(52) U.S. Cl. CPC B22F 9/24; B22F 7/08; G01N 27/327; B22F 2301/255; B22F 2303/20; B22F 2304/10

(57) ABSTRACT

A sensor is disclosed. The sensor comprises a substrate; and a gold pattern attached to the substrate, wherein the gold pattern is made from a plurality of repeating units, each unit is made from at least one line having a width of between 100 to 500 nm and a length of between 1 to 50 microns, and wherein a first distance between two neighboring units is between 50 to 1000 nm, and wherein all lines in the pattern, are originated from a monocrystalline gold, therefore, have the same crystallographic orientation with respect to the substrate.

### CROSS-REFERENCE TO RELATED APPLICATIONS

This application is a National Phase of PCT Patent Application No. PCT/IL2022/050409 having International filing date of 19 Apr. 2022, which claims the benefits of priority of U.S. Provisional Patent Application No. 63/176,407 filed Apr. 19, 2021, the contents of which are incorporated herein by reference in their entirety.

### FIELD OF THE INVENTION

The invention relates generally to the field of gold microparticles and articles, such as sensors, comprising same.

### BACKGROUND

The solution-based chemical process has been established decades ago as a successful strategy for the bottom-up growth of nanomaterials (nano-plasmonic and semiconductors). Today, it is the main synthetic route for fabricating almost all types of nanomaterials with well-controlled shapes, sizes, compositions, and disparity.

The formation of 2D monocrystalline gold flakes is particularly appealing as a basis for precise plasmonic nanostructures for the transport of both optical signals without grain boundaries or defects and for associated strongly enhanced local fields with marginal ohmic losses. Thus, these 2D structures combine the superior technical advantages of both photonics and electronics on the same chip.

However, to the best of our knowledge, a reliable and cost-efficient procedure for manufacturing of the 2D monocrystalline gold flakes has not been previously described. Thus, there is an unmet need for a method of manufacturing micrometer sized 2D monocrystalline gold flakes, being suitable inter alia for use as plasmonic surfaces.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

Some aspects of the invention are directed to a sensor comprising: a substrate; and a gold pattern attached to the substrate, wherein the gold pattern is made from a plurality of repeating units, each unit is made from at least one line having a width of between 100 to 500 nm and a length of between 1 to 50 microns, and wherein a first distance between two neighboring units is between 50 to 1000 nm, and wherein all lines in the pattern, are originated from a monocrystalline gold microparticle, therefore, have the same crystallographic orientation with respect to the substrate.

In some embodiments, each unit comprises more than two lines or more than two segments in a line, and wherein a second distance between the two lines or the two segments is between 1 to 50 nm.

In some embodiments, the second distance is determined based on a required optical absorption of specific wavelengths. In some embodiments, the second distance is determined as to cause the sensor to generate a surface plasmon polariton at a specific wavelength.

In some embodiments, the first distance is determined based on a required optical absorption of specific wavelengths. In some embodiments, the pattern is a three-dimensional (3D) pattern. In some embodiments, the thickness of the pattern is between 10 to 100 nm. In some embodiments, the substrate is a dielectric substrate selected from, silicon wafer, glass, polymer, silica, and any ceramic material.

In some embodiments, the sensor further comprises an antibody attached to the gold pattern. In some embodiments, the antibody is covalently attached to the gold pattern via a sulfuric bond.

In some embodiments, the sensor further comprises a molecule bound to a surface of said gold pattern, the molecule is selected from: a substituted or an unsubstituted mercaptoalkyl, mercaptoaryl, mercaptoalkaryl, dialkyl sulfide, diaryl sulfide or a combination thereof.

In one aspect of the invention, there is provided a microparticle, wherein at least 99.999% by weight of the microparticle consist of a monocrystalline gold; a surface area of the microparticle particle is between 0.006 mm² and 1 mm; a thickness of the microparticle particle is between 10 and 100 nm.

In one embodiment, a width dimension or a length dimension of the microparticle is between 0.5 um and 300 um.

In one embodiment, microparticle is a two-dimensional (2D) microparticle.

In one embodiment, the microparticle is in a form of a uniform layer.

In one embodiment, uniform layer is a monolayer.

In one embodiment, a surface roughness of the uniform layer is between 0.1 and 2 nm.

In one embodiment, a permittivity of the microparticle is between 0.3 and 0.7.

In one embodiment, the microparticle further comprises a molecule bound to a surface of the microparticle.

In one embodiment, the molecule comprises a thiol group comprising a substituted or an unsubstituted mercaptoalkyl, mercaptoaryl, mercaptoalkaryl, dialkyl sulfide, diaryl sulfide or a combination thereof.

In one embodiment, the molecule comprises a plurality of molecules in a form of a monolayer.

In one embodiment, bound is via a covalent bond, or via an ionic bond.

In one embodiment, the microparticle is characterized by an adhesiveness to a substrate selected from silicon, glass, a metal, a dielectric material, and a semiconductor including any combination thereof.

In one embodiment, the microparticle is characterized by a shape selected from a tringle, a hexagon, a truncated triangle, or any combination thereof.

In one embodiment, the microparticle is configured to generate a surface plasmon polariton.

In another aspect, there is an article comprising a substrate in contact with the microparticle of the invention.

In one embodiment, the article comprises a monolayer of molecules bound to a surface of the microparticle.

In one embodiment, the molecules comprising any one of a substituted or an unsubstituted mercaptoalkyl, mercaptoaryl, mercaptoalkaryl, dialkyl sulfide, diaryl sulfide or a combination thereof.

In one embodiment, the article is configured for generating a direct current triboelectricity.

In another aspect, there is a method of manufacturing the microparticle of the invention, comprising a. providing a reaction mixture comprising a gold salt, a reducing agent, and a solvent; b. providing the reaction mixture to a temperature of between 25 and 150° C. for a time period of between 60 min and 5 h; c. aging the reaction mixture for a time period of between 60 min and 7 days, thereby obtaining the microparticle; wherein: the gold salt comprises Au(III) cation; the reducing agent is capable of reducing the Au(III) cation to an elemental state; and wherein a concentration of the gold salt within the reaction mixture is between 0.03 and 0.3 mM.

In one embodiment, a concentration of the reducing agent within the reaction mixture is between 0.19 and 10 M.

In one embodiment, the reducing agent comprises aniline.

In one embodiment, the solvent comprises a polar solvent.

In one embodiment, the polar solvent comprises a glycol, an alcohol or both.

In one embodiment, the gold salt comprises HAuCl₄, including any hydrate thereof.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-1D are micrographs, representing SEM images of nano- and microstructures synthesized by implementing various gold precursor (Auₚ):Aniline molar ratios of 0.1:1 resulting in gold nano-plates (1A); 1:1 resulting in gold micro-plates (1B); 10:0.3 resulting in gold nano-wires (1C); and gold icosahedra 10:1 (1D). The reaction times were as follows: 3 h (1A); 3 h (1B); 5 h (2C); and 0.5 h (1D).

FIGS. 2A-2D are micrographs, representing SEM images of nano- and microplates synthesized by implementing various gold precursor (Auₚ):Aniline concentrations of 0.1:0.1M (2A); 10:10M (2B); 20:20 M (2C); and 1:1M (2D).

FIGS. 3A-3F are micrographs, representing SEM images of microplates synthesized at varying the reaction times: 3 hours (3A); 1 day (3B) and 7 days (3C); and at varying reaction temperature of 25° C. (3D), 50° C. (3E) and 100° C. (3F).

FIGS. 4A-4D are micrographs, representing SEM images of microplates.

FIG. 5 is a graph representing average surface area as a function of gold precursor concentration calculated according to total reaction volume after addition of aniline (black curve) and scaling up or down the reaction molar ratio of constitutes by a certain factor (blue curve).

FIG. 6 is a graph representing average area of gold plates during the first 3 hours of the reaction (black curve) and in a 7 day period (black curve). Inset is the average area as a function of temperature. Gold concentration is 2.76 mg/ml.

FIGS. 7A-7F are micrographs, representing SEM images of Gold nano/microplates synthesized by the aniline assisted polyol method as a function of gold precursor concentration: (7A-7F) 0.27, 1.35, 2.76, 27.6, 83.99 and 54.4 mg/mL respectively.

FIG. 8A is an illustration representing various reaction conditions required for controlling the size range of gold-plates.

FIG. 8B is a flowchart of a method of manufacturing the microparticle according to some embodiments of the invention.

FIG. 9 is an illustration of a sensor according to some embodiments of the invention.

FIGS. 10A, 10B and 10C are illustrations of gold monocrystalline patterns according to some embodiments of the invention.

FIG. 11 is an illustration of units of a pattern according to some embodiments of the invention.

FIG. 12 is an illustration of a sensor with antibodies according to some embodiments of the invention.

FIG. 13 is an illustration an attachment of an analyte of interest to an antibody included in a sensor.

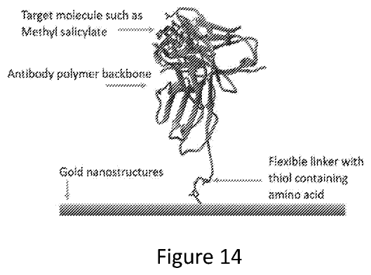

### DETAILED DESCRIPTION

The present invention, in one aspect thereof, relates to a composition comprising one or more monocrystalline gold microparticle.

In another aspect, the present invention relates to a substrate in contact with the one or more monocrystalline gold microparticle. In some embodiments, the monocrystalline gold microparticle further comprises a monolayer of molecules bound thereto. In some embodiments, there is an apparatus comprising the substrate in contact with the one or more monocrystalline gold microparticle. In some embodiments, the apparatus is for generating a direct current triboelectricity.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### The Microparticle

In one aspect of the invention, there is provided a microparticle, wherein: at least 99% by weight of the microparticle consist of a monocrystalline gold; a surface area of the is between 0.006 mm² and 1 mm; a thickness of the microparticle is between 10 and 100 nm.

In some embodiments, the microparticle of the invention consists substantially of monocrystalline gold in an elemental state. In some embodiments, the microparticle of the invention is in a form of a solid.

In some embodiments, at least 99%, at least 99.5%, at least 99.9%, at least 99.99%, at least 99.999%, by weight of the microparticle of the invention is or comprises gold in an elemental state.

In some embodiments, at least 99%, at least 99.5%, at least 99.9%, at least 99.99%, at least 99.999%, by weight of the microparticle of the invention is or comprises monocrystalline gold.

As used herein, the term “monocrystalline” refers to a solid material (usually metallic material) in which the crystal lattice of the entire sample is continuous and unbroken to the edges of the sample, with no grain boundaries. One skilled artisan will appreciate, that the monocrystalline material is devoid of plurality of grains, causing defects associated with grain boundaries (cavities filled with an ambient gas). Monocrystalline materials are uniform within the entire bulk sample, generating a continuous uniform surface being devoid of empty space and/or impurities.

In some embodiments, the microparticle of the invention solely comprises crystalline gold. In some embodiments, the plurality of gold atoms within the microparticle of the invention are substantially in a crystalline form. In some embodiments, the microparticle of the invention is substantially devoid of amorphous gold atoms.

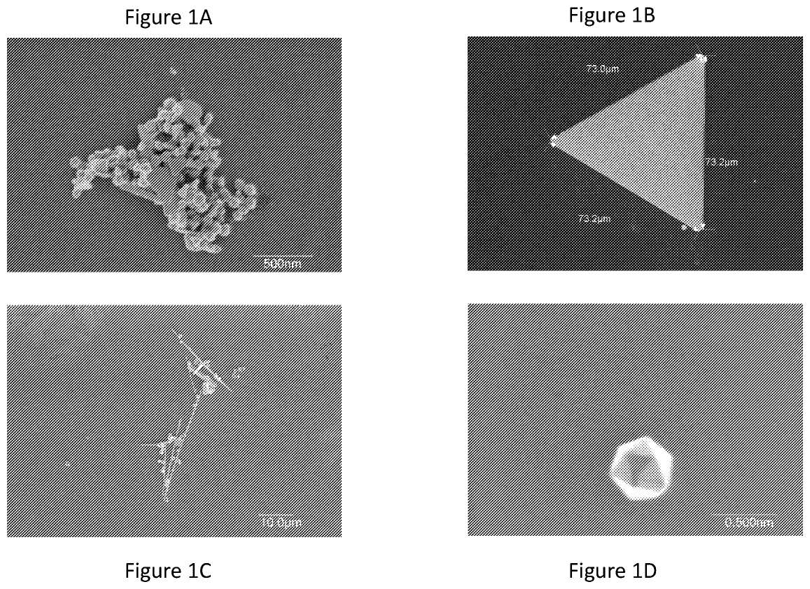

Some non-limiting examples of such particles are given and discussed below in the Example section with respect to FIGS. 1 to 7.

In some embodiments, the microparticle of the invention is in a form of a flake or a plate. In some embodiments, the microparticle of the invention is in a form of a two-dimensional (2D) microparticle or microplate. In some embodiments, the microparticle of the invention is in a form of a layer.

As used herein, the term “two-dimensional microparticle” relates to any material consisting of a single layer (sometimes several tens of layers) of atoms or a stack of such layers.

In some embodiments, the microparticle (also referred to herein as “2D microparticle” or “layer”) is characterized by a uniform thickness. In some embodiments, the microparticle is characterized by a non-uniform thickness. In some embodiments, the microparticle of the invention is in a form of a uniform layer. In some embodiments, the microparticle of the invention is in a form of a homogenous layer. In some embodiments, the microparticle of the invention is a monolayer of gold atoms. In some embodiments, the outer surface of the microparticle of the invention is substantially uniform (or flat). In some embodiments, the outer surface of the microparticle of the invention is substantially homogenous. In some embodiments, the outer surface of the microparticle of the invention is substantially composed of a monolayer of gold atoms.

In some embodiments, a surface roughness of the microparticle or of the uniform layer described herein is between 0.1 and 1 nm, between 0.1 and 0.3 nm, between 0.3 and 0.5 nm, between 0.5 and 0.7 nm, between 0.7 and 1 nm, between 1 and 2 nm, between 2 and 5 nm, including any range therebetween.

In some embodiments, a surface roughness of the microparticle or of the uniform layer described herein is less than 2 nm, less than 1.5 nm, less than 1.3 nm, less than 1.2 nm, less than 1.1 nm, including any range therebetween.

In some embodiments, at least 80%, at least 85%, at least 90%, at least 95%, at least 99% of the outer surface of the microparticle is characterized by a surface roughness between 0.1 and 1 nm, including any range therebetween.

In some embodiments, the microparticle comprises between 1 and 100, between 1 and 10, between 10 and 50, between 50 and 100, between 100 and 200, between 200 and 500, between 500 and 1000 atomic layers, including any range therebetween.

In some embodiments, the microparticle of the invention has an average thickness from 10 nm to 100 um, from 1 nm to 5 nm, from 5 nm to 10 nm, from 10 nm to 20 nm, from 20 nm to 30 nm, from 30 nm to 50 nm, from 50 nm to 100 nm, including any range therebetween.

In some embodiments, the microparticle of the invention is stable upon storage at a temperature below the melting point of gold in the ambient atmosphere.

As used herein, the term “stable” is referred to the ability of the composition to maintain at least 80%, at least 85%, at least 90% of its structural intactness and/or its chemical composition. In some embodiments, the stable composition is capable to maintain its crystalline structure upon storage. In some embodiments, the stable composition is capable to maintain at least 80%, at least 85%, at least 90% of its dimension (e.g. geometrical shape, 2D shape, thickness, area, perimeter or a combination thereof) upon storage.

In some embodiments, the term “storage” refers to a time period of 1 day and 10 years, including any range between. In some embodiments, the term “storage” refers to storage conditions as described herein.

In some embodiments, the microparticle is substantially devoid of an additional metal, and/or a slat thereof. In some embodiments, the microparticle is substantially devoid of a gold salt (e.g. HAuCl₄). In some embodiments, the microparticle is substantially devoid of an organic polymer (e.g. PANI). In some embodiments, the microparticle is substantially devoid of a solvent (e.g. an aqueous solvent, an organic solvent such as an alcohol, a glycol or a combination thereof).

In some embodiments, the microparticle of the invention is characterized by an average surface area of between 0.006 and 1 mm², between 0.006 and 0.01 mm², between 0.01 and 0.05 mm², between 0.05 and 0.1 mm², between 0.1 and 0.2 mm², between 0.2 and 0.3 mm², between 0.3 and 0.5 mm², between 0.5 and 0.7 mm², between 0.7 and 1 mm², including any range therebetween.

In some embodiments, the microparticle of the invention is characterized by an average surface area of at least 0.06 mm², at least 0.08 mm², at least 0.1 mm², at least 0.2 mm², including any range therebetween.

In some embodiments, the microparticle of the invention has a surface area of between 0.006 mm² and 1 mm; and a thickness of between 10 and 100 nm, including any range therebetween. In some embodiments, at least 99%, at least 99.5%, at least 99.9%, at least 99.99%, at least 99.999% by weight of the microparticle consist of a monocrystalline gold; and characterized by (i) a surface area of between 0.006 mm² and 1 mm; and (ii) an average thickness of between 10 and 100 nm, including any range therebetween.

In some embodiments, the microparticle of the invention has any geometrical shape (e.g. 2D shape). In some embodiments, the microparticle of the invention has a shape selected form a triangle, a truncated triangle, a rectangle, a hexagon, a pentagon, a circle, am ellipse, or a combination thereof. In some embodiments, the microparticle of the invention is devoid of a defined geometrical shape.

In some embodiments, the microparticle of the invention has a planar shape. In some embodiments, the microparticle of the invention has a planar geometry, being substantially flat. In some embodiments, the microparticle of the invention has a planar geometry, being substantially in a single plane. In some embodiments, the shape of the composition is controllable.

In some embodiments, the microparticle of the invention has at least one horizontal dimension of between 0.5 um and 300 um, between 0.5 um and 1 um, between 1 um and 10 um, between 10 um and 50 um, between 50 um and 100 um, between 100 um and 200 um, between 200 um and 300 um, including any range therebetween.

In some embodiments, the microparticle of the invention is in a form of a polygon. In some embodiments, each side of the polygon has a length of between 0.5 um and 300 um, between 0.5 um and 1 um, between 1 um and 10 um, between 10 um and 50 um, between 50 um and 100 um, between 100 um and 200 um, between 200 um and 300 um, including any range therebetween. In some embodiments, each side of the polygon has the same or different length.

In some embodiments, the microparticle of the invention is in a form of a triangle, a truncated triangle, a hexagon or both.

In some embodiments, the microparticle of the invention has a width dimension or a length dimension of between 0.5 um and 300 um, between 0.5 um and 1 um, between 1 um and 10 um, between 10 um and 50 um, between 50 um and 100 um, between 100 um and 200 um, between 200 um and 300 um, including any range therebetween.

In some embodiments, the microparticle of the invention is characterized by permittivity of between 0.3 and 0.7, between 0.3 and 0.5, between 0.5 and 0.6, between 0.6 and 0.7, between 0.7 and 0.8, including any range therebetween. As used herein, the term “permittivity” relates the electric polarizability of the microparticle.

In some embodiments, the microparticle of the invention is configured to generate a surface plasmon polariton. In some embodiments, the microparticle of the invention is capable of sustaining surface plasmon resonance. In some embodiments, the microparticle of the invention is capable of supporting or inducing formation of a surface plasmon polariton upon contacting the microparticle with light of an appropriate wavelength.

One skilled in the art will appreciate, that light of an appropriate wavelength incident on the microparticle of the invention interacts with the gold electrons to form electron-plasma oscillations along the outer surfaces of microparticle. These quantized electron-plasma oscillations are referred to as surface plasmon polaritons (“SPPs”), and the oscillations produce corresponding electron excitations that exist on the surface of the microparticles. The SPPs have longitudinal and transverse electromagnetic field components. The magnetic field component is approximately parallel to the outer surface, while the electric field component is perpendicular to the outer surface and has a high intensity within a few tens of nanometers from the outer surface (transverse magnetic-waves).

In some embodiments, there is a composition comprising a plurality of microparticles of the invention. In some embodiments, composition is a solid composition. In some embodiments, the plurality of microparticles are distinct within the composition. In some embodiments, the composition comprises a plurality of distinct microparticles. In some embodiments, the composition is substantially devoid of aggregated particles (e.g. dimers, trimers or multi-particle aggregates).

In some embodiments, the microparticle of the invention is characterized by an adhesiveness to a substrate. In some embodiments, the microparticle of the invention is capable of binding to a substrate. In some embodiments, the microparticle of the invention is characterized by binding affinity to the substrate. In some embodiments, adhesiveness and/or binding comprises non-covalent interaction (such interactions are well-known in the art in include inter alia Van-der Waals forces, London forces, dipole-dipole-interactions, pi-stacking etc.).

In some embodiments, the substrate is selected from silicon, glass substrate including any derivatives thereof (e.g. indium-tin oxide coated glass or ITO glass; fluorine dopped tin oxide coated glass FTO), a metal substrate, a dielectric material, and a semiconductor including any mixture thereof.

In some embodiments, the dielectric material comprises a material selected from a ferroelectric material, a ferromagnetic material, a piezoelectric material, a pyroelectric material and an insulator or any combination thereof. Several dielectric materials are well-known in the art.

In some embodiments, the microparticle of the invention further comprises a molecule bound to an outer surface of the microparticle. In some embodiments, at least a portion of outer surface of the microparticle of the invention is bound to a plurality of molecules.

In some embodiments, the molecule has an affinity to gold atoms. In some embodiments, the molecule has a binding affinity to gold atoms. In some embodiments, the molecule has reactivity to gold atoms. In some embodiments, the molecule is capable of forming a bonding interaction with a gold atom and/or outer surface of the microparticle of the invention. In some embodiments, the molecule is capable of forming an electrostatic interaction, a covalent bond, a coordinative bond including any combination thereof, with a gold atom and/or outer surface of the microparticle of the invention. In some embodiments, the molecule is capable of forming a stable bond with gold atom and/or outer surface of the microparticle of the invention, wherein stable refers to the chemical stability of the bond under ambient conditions, for a time period of between 1 day and 1 year including any range between.

In some embodiments, the molecule comprises a functional group capable of forming a stable bond with gold atom and/or outer surface of the microparticle of the invention. In some embodiments, the functional group capable of forming a stable bond with gold atom is a thiol group.

In some embodiments, the molecule is or comprises a thiol group. In some embodiments, the molecule is or comprises a sulfide. In some embodiments, the molecule is or comprises a substituted or an unsubstituted mercaptoalkyl, mercaptoaryl, mercaptoalkaryl, dialkyl sulfide, diaryl sulfide or a combination thereof.

In some embodiments, the molecule is chemisorbed to the microparticle of the invention. In some embodiments, the thiol group is covalently bound to the microparticle. In some embodiments, the molecule is covalently bound to the microparticle via the thiol group. In some embodiments, the molecule is bound to the microparticle via an ionic bond. In some embodiments, the thiol group of the molecule bound to the microparticle is in an ionized (or deprotonated) form. In some embodiments, the thiol group forms a stable Au(0) thiolate bond with gold atom of the microparticle. In some embodiments, the thiol group forms a stable gold-thiolate complex with gold atom of the microparticle. In some embodiments, the thiol group of the molecule is bound to the gold atom via a coordinative bond, wherein the thiol (or thiolate) is an anionic ligand.

In some embodiments, the molecule comprises an (C5-C50) mercaptoalkyl. In some embodiments, the molecule comprises an (C5-C30) mercaptoalkyl (e.g. octanthiol, decanthiol, octadecanthiol, etc.). In some embodiments, the molecule comprises an aromatic thiol. In some embodiments, the molecule comprises benzenthiol substituted by any of haloalkyl (e.g. CF3), alkylamine (e.g. dimethylamine, trimethylammonium), alkyl (e.g. tert-butyl) or any combination thereof.

In some embodiments, the molecule comprises an antibody. In some embodiments, the antibody has an affinity to an analyte (e.g. an analyte detectable by the sensor of the invention). In some embodiments, the antibody is bound to the gold atom and/or outer surface of the microparticle via a covalent bond or via a non-covalent bond (e.g. by physisorption).

In some embodiments, the molecule comprises a thiol group bound to the antibody via a linker or a spacer. In some embodiments, the antibody is a specific antibody, having an enhanced binding affinity to the analyte, as compared to a control (e.g. a small molecule which is not the analyte). In some embodiments, enhanced is by at least 100%, at least 1000%, at least 50 times, at least 100 times, at least 1000 times, at least 10000 times, at least 100000 times, at least 1000000 times, at least 1000000000 times greater binding affinity to the analyte, as compared to the control, including any range between. In some embodiments, the sensor of the invention comprising an antibody bound to the gold surface (i.e. to the surface of the microparticle), is characterized by an enhanced sensitivity to a specific analyte, as compared to a control, or as compared to an analogous sensor devoid of the antibody; and wherein enhanced is as described herein.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelid, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulfide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the linker is or comprises a spacer (e.g., a natural and/or unnatural amino acid, alkyl, an amide bond, an ester bond, a thioester bond, a urea bond, including any derivative or a combination thereof). In some embodiments, the spacer comprises any one of: a (C5-C50) mercaptoalkyl (e.g. octanthiol, decanthiol, octadecanthiol, etc.), an aromatic thiol, a benzenethiol substituted by any of haloalkyl (e.g. CF3), alkylamine (e.g. dimethylamine, trimethylammonium), alkyl (e.g. tert-butyl) or any combination thereof.

In some embodiments, the linker comprises a polyglycol ether (e.g., polyethylene glycol (PEG)), a polyester, a polyamide, a polyamino acid, a peptide and/or a derivative thereof or any combination thereof. In some embodiments, the linker of the invention is characterized by Mw of between 100 and 5000 Da including any range between.

In some embodiments, the polyamino acid or a derivative thereof comprises between 2 and 100 amino acids, between 4 and 50, between 4 and 100, between 5 and 50, between 5 and 50, between 4 and 20, between 4 and 30, between 4 and 40, between 5 and 20, between 5 and 30, between 5 and 40, between 6 and 50, between 6 and 30, between 6 and 40, between 6 and 20, between 8 and 50, between 8 and 30, between 8 and 20, between 8 and 40, including any range between.

The terms “peptide”, and “polyamino acid” are used herein interchangeably. The terms “peptide”, “polypeptide” and “protein” as used herein encompass native peptides, peptide derivatives such as beta peptides, peptidomimetics (typically including non-peptide bonds or other synthetic modifications,) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

The term “derivative” or “chemical derivative” includes any chemical derivative of the polypeptide having one or more residues chemically derivatized by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and Dab, Daa, and/or ornithine (O) may be substituted for lysine.

In addition, a peptide derivative can differ from the natural sequence of the peptide of the invention by chemical modifications including, but are not limited to, terminal-NH₂ acylation, acetylation, or thioglycolic acid amidation, and by amidation of the terminal and/or side-chain carboxy group, e.g., with ammonia, methylamine, and the like. Peptides can be either linear, cyclic, or branched and the like, having any conformation, which can be achieved using methods known in the art.

In some embodiments, the linker of the invention further comprises a spacer (e.g., a natural and/or unnatural amino acid, optionally substituted alkyl, an amide bond, an ester bond, disulfide bond, a thioester bond, a urea bond, including any derivative or a combination thereof). In some embodiments, the linker of the invention comprises a click reaction product (e.g., a covalent linkage such as a cyclization reaction product, and/or a succinimide-thioether moiety formed via a click reaction).

Click reactions are well-known in the art and comprise inter alia Michael addition of maleimide and thiol (resulting in the formation of a succinimide-thioether); azide alkyne cycloaddition; Diels-Alder reaction (e.g., direct and/or inverse electron demand Diels Alder); dibenzyl cyclooctyne 1,3-nitrone (or azide) cycloaddition; alkene tetrazole photoclick reaction etc.

In some embodiments, the antibody is bound to the gold surface via a side chain of at least one cysteine. In some embodiments, the cysteine is N-terminal or C-terminal cysteine. In some embodiments, the antibody is bound to the gold surface via a peptide linker, wherein the peptide linker comprises a terminal cysteine. In some embodiments, the peptide linker is a part of the amino acid sequence of the antibody. In some embodiments, the peptide linker is located at the C-terminus or at N-terminus of the amino acid sequence of the antibody.

In some embodiments, the linker comprises a bifunctional moiety capable of reacting with the antibody and with a spacer covalently bound to the gold surface.

In some embodiments, the antibody is bound to the gold surface via a click reaction product, or via a disulfide bond. For example, a linker or a spacer comprises a thio group at the first end and a reactive group at the second end, wherein the reactive group is capable of reacting with the antibody via click chemistry. Exemplary reactive groups comprise inter alia NHS ester (capable of reacting with an amine of the antibody), maleimide (capable of reacting with a thiol of the antibody), SPDP (including any derivative thereof, capable of reacting with a thiol of the antibody to form a disulfide bond, etc.). In some embodiments, the antibody is modified with a moiety (e.g. a peptide linker having a terminal lysine or cysteine, azide, alkyne, dibenzyl cyclooctyne, alkene, tetrazine, SPDP group, etc.) wherein the moiety is capable of reacting with the reactive group of the linker.

In some embodiments, the plurality of molecules bound to the outer surface of the microparticle are in a form of a monolayer. In some embodiments, the plurality of molecules bound to the outer surface of the microparticle, form a unimolecular layer on top of the microparticle.

### Manufacturing Methods

In another aspect of the invention, there is provided a method of manufacturing the composition of the invention, wherein the composition is as described herein.

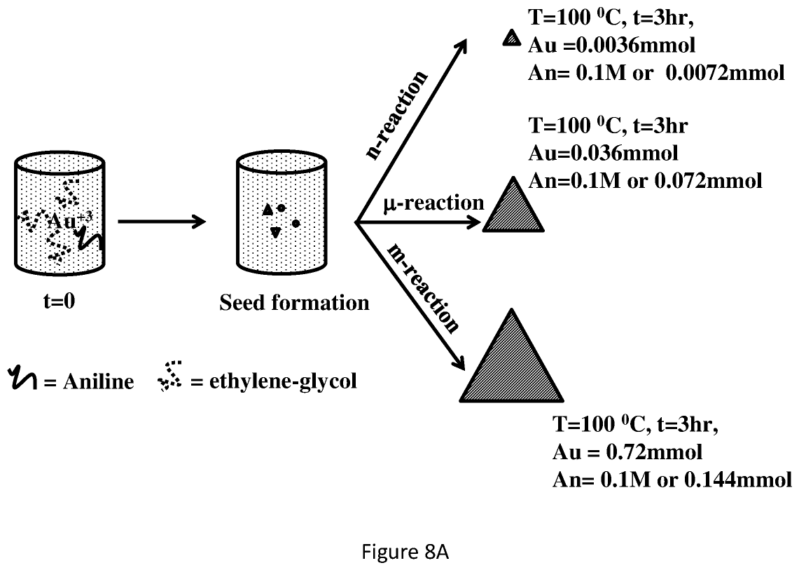

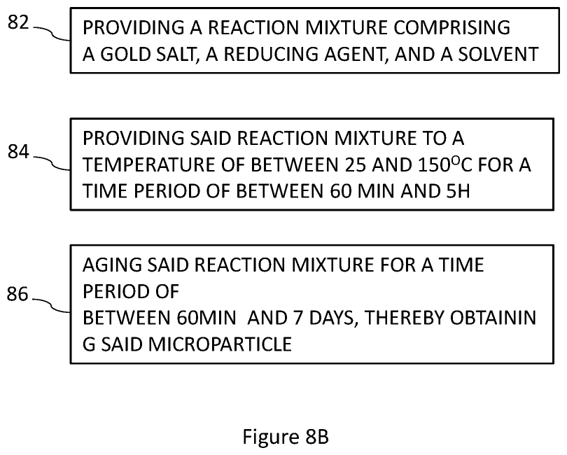

Reference is now made to FIGS. 8A and 8B which are illustration of a manufacturing method and a flowchart of method of manufacturing the microparticle according to some embodiments of the invention.

In some embodiments, the method of manufacturing a plurality of microparticles of the invention, comprises the steps of:
(Step 82). providing a reaction mixture comprising a gold salt, a reducing agent, and a solvent;
(Step 84) providing the reaction mixture to conditions suitable for reducing of the gold salt to elemental gold; and
(Step 86) aging the reaction mixture for a time period of between 60 min and 7 days, thereby obtaining the monocrystalline gold microparticles of the invention. In some embodiments, the gold salt is a precursor of elemental gold. In some embodiments, the precursor is reducible by the reducing agent. In some embodiments, the reducing agent has a reduction potential sufficient for reducing the precursor.

In some embodiments, the conditions suitable for reducing of the gold salt to elemental gold comprise a temperature of between 25 and 150° C., between 25 and 50° C., between 50 and 70° C., between 70 and 90° C., between 90 and 100° C., between 100 and 130° C., between 130 and 150° C., including any range between.

In some embodiments, the conditions suitable for reducing of the gold salt (Au(III) cation) to elemental gold comprise a time period (e.g. reaction time) of between 1 and 5 h, between 1o min and 0.5 h, between 0.5 and 1 h, between 1 and 2 h, between 2 and 3 h, between 3 and 5 h, including any range between.

In some embodiments, the gold salt comprises Au(III) cation. In some embodiments, the gold salt comprises an anion (e.g. halide). In some embodiments, the gold salt is or comprises HAuCl₄, including any hydrate thereof.

In some embodiments, a molar concentration of the gold salt within the reaction mixture is between 0.03 and 3 mmol/L, 0.03 and 0.05 mmol/L, 0.05 and 0.1 mmol/L, 0.1 and 0.2 mmol/L, 0.2 and 0.3 mmol/L, 0.3 and 0.5 mmol/L, 0.5 and 0.7 mmol/L, 0.7 and 1 mmol/L, including any range between.

In some embodiments, a molar concentration of the reducing agent within the reaction mixture is between 0.1 and 10 mol/L, between 0.1 and 0.2 mol/L, between 0.2 and 0.3 mol/L, between 0.3 and 0.5 mol/L, between 0.5 and 1 mol/L, between 1 and 3 mol/L, between 3 and 5 mol/L, between 5 and 10 mol/L, including any range between.

In some embodiments, a molar ratio between the reducing agent and the gold salt within the reaction mixture is between 50:1 and 1:1, between 50:1 and 30:1, between 30:1 and 20:1, between 20:1 and 10:1, between 10:1 and 5:1, between 5:1 and 1:1, between 1:1 and 1:2, between 1:2 and 1:5, including any range between.

In some embodiments, a molar ratio between the reducing agent and the gold salt within the reaction mixture is between 20:1 and 10:1. In some embodiments, a molar concentration of the reducing agent within the reaction mixture is between 0.1 and 0.2 mol/L, a molar concentration of the gold salt within the reaction mixture is 0.5 and 1 mmol/L.

In some embodiments, reducing agent comprises aniline. In some embodiments, reducing agent comprises polyaniline or a derivative thereof.

In some embodiments, the solvent comprises a polar solvent. In some embodiments, the polar solvent comprises a glycol (e.g. ethyleneglycol, propyleneglycol) an alcohol (e.g. methanol, ethanol, propanol, butanol) or both.

In some embodiments, step c (aging) is performed at the same temperature as the step b. In some embodiments, step c is performed for a time period sufficient for the formation of microparticle of the invention. In some embodiments, step c is performed for a time period ranging between 60 min and 7 days (d), between 10 min and 1 hour (h), between 1 and 10 h, between 10 and 24 h, between 1 d and 2 d, between 2 d and 5 d, between 5 d and 10 d, including any range between.

In some embodiments, the time period of step c predetermines the size of the resulting microparticles. In some embodiments, the ratio between the reducing agent and the gold salt; and the time period of step c predetermines the size (e.g. at least one dimension as described herein) of the resulting microparticles.

### Sensor

According to an aspect of some embodiments of the present invention, there is provided an article, such as a sensor for sensing an analyte of interest. A non-limiting example, for such an analyte of interest, is methyl salicylate. In some embodiments, the sensor may be based on surface-enhanced Raman scattering (SERS) which applied using non-resonant near-infrared excitation. The SERS technique generally profits from the strong increase of the intrinsically weak Raman signals caused by the presence of nanosized metallic structures, for example when the target molecule is attached to colloidal silver and gold clusters. When only a small sample amount is available, surface enhancement methods offer promising opportunities in biology, medicine, and pharmacy, and allow studies of the relationships between the structure and function of molecule.

In some embodiments, the spectroscopic detection is based on the inelastic scattering of molecular vibrations (Raman spectroscopy), plasmonic nanostructures can extremely enhance the signal strength as reported by the former research group, we will use the SERS, as a method which detects the Raman signature of analytes for which a signal enhancement through nano-structuring can occur so that even extremely low analyte concentrations can be detected.

In some embodiments, such a sensor may include a gold pattern attached to a substrate. The gold pattern is originated from a monocrystalline gold microparticle. In a non-limiting example, the gold pattern is originated from a monocrystalline gold particle according to embodiments of the invention, discussed herein. However, as should be understood by one skilled in the art, the invention is not limited to specific monocrystalline gold pattern or a method of making a monocrystalline gold pattern on a substrate, as disclosed herein.

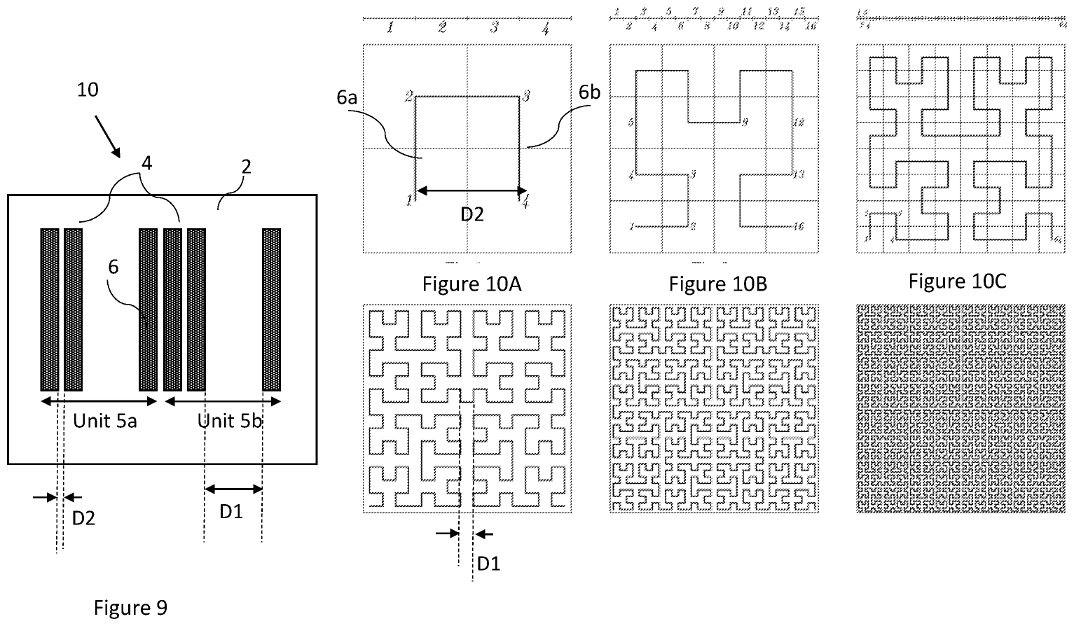

Reference is now made to FIG. 9, which is an illustration of a sensor according to some embodiments of the invention. A sensor 10 may include a substrate 2 and a gold pattern 4 attached to substrate 2. In some embodiments, substrate 2 is a dielectric substrate selected from, silicon wafer, glass, polymer, silica, and any ceramic material or the like.

In some embodiments of the present invention, substrate 2 have incorporated on at least a portion thereof the microparticle of the invention. In some embodiments, substrate 2 is in contact with a layer comprising a plurality of microparticles. In some embodiments, the substrate is bound or adhered to a plurality of microparticles, wherein bound or adhered is via non-covalent bond. In some embodiments, the substrate is as described hereinabove. In some embodiments, since the monocrystalline gold microparticle is fabricated separately from the substrate, any dialectic substrate can be used, regardless of it's crystalline/amorphous structure. In some embodiments, the dialectic substrate is selected based on required optical properties, for example, the refractive index, the absorption and/or reflection coefficients and the like.

By “a portion thereof” it is meant, for example, a surface or a portion thereof, and/or a body or a portion thereof, of a solid or a semi-solid substrate.

In some embodiments, substrate 2 is bound to the microparticle, wherein the outer surface of the microparticle is in contact with or bound to a molecule comprising a thiol group, as described herein. In some embodiments, the substrate is bound to the microparticle, wherein the outer surface of the microparticle is in contact with or bound to a monolayer of thiol-based molecules (or sulfides), wherein the thiol-based molecules optionally comprise an antibody bound thereto, as described hereinabove. In some embodiments, the substrate consists essentially of monocrystalline gold and is devoid of organic and/or inorganic molecules bound thereto.

In some embodiments, sensor 10 of the invention comprises a dielectric material (e.g. glass, silicon, etc.) as substrate 2 in contact with the microparticle of the invention, wherein the outer surface of microparticle is bound to a monolayer of the molecules as described herein. In some embodiments, the microparticle is bound to a monolayer of mercaptoaryl molecules. In some embodiments, the microparticle is bound to a monolayer of benzenthiol substituted by an elector-withdrawing group (e.g. CF3). In some embodiments, the monolayer is configured to attenuate tunneling current. In some embodiments, the monolayer is characterized by a tunneling attenuation current ranging between 1 nA and 100 mA including nay range between.

In some embodiments, sensor 10 of the invention is configured to be electrically charged by friction. In some embodiments, the outer surface of the microparticle is configured to be electrically charged by friction. In some embodiments, the article of the invention is configured to generating a direct current (DC) triboelectricity upon friction of the outer surface of the microparticle with an electrode.

In some embodiments, gold pattern 4 is attached to substrate 2. In some embodiments, the gold nanoparticle is first attached to the substrate as disclosed hereinabove. Alternatively, a gold nanoparticle may be deposited, grown, etc. on top of substrate 2. In some embodiments, pattern 4 may be formed using any known ablation method. For example, for high-resolution nanostructuring via Python scripting.

A non-limiting example, a pattern design was modeled by full-field electromagnetic modeling based on finite elements. Thereby, the local field enhancements can be optimized while the patterning time is kept as short as possible. The location of each gold nanoparticle was identified on substrate 2 (e.g., a silicon). The locations were identified by designing an optical mask with specific markers (as a road map) to follow the position of the deposited 2D gold nanoparticle (the mask fabricated by standard optical lithography). After this, markers were realized on Si substrate by metal evaporation.

In some embodiments, the gold pattern 4 is made from a plurality of repeating units 5A, 5B, each unit is made from at least one line 6 having a width of between 100 to 500 nm and a length of between 1 to 50 microns, and wherein a first distance D1 between two neighboring units 5A and 5B is between 50 to 1000 nm. In some embodiments, all lines in pattern 4, are originated from a monocrystalline gold microparticle, therefore, have the same crystallographic orientation with respect to the substrate. In some embodiments, each unit 5A and 5B may include a single line and first distance D1 between two neighboring lines is between 1 to 50 nm.

In some embodiments, first distance D1 between two neighboring units 5A and 5B is between 50 to 100 nm, between 50 to 200 nm, between 150 to 300 nm, between 300 to 700 nm, and between 500 to 1000 nm, or any value in between.

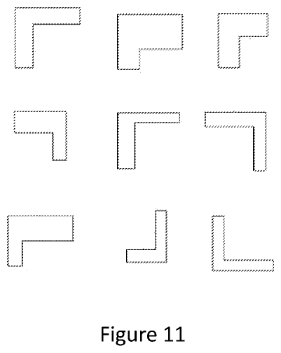

In some embodiments, each unit 5A and 5B may include more than two lines 6 or more than two segments 6a and 6b a line, as, illustrated in FIG. 10A and wherein a second distance D2 between the two lines or the two segments is between 1 to 50 nm, for example, between 10 to 50 nm, between 1 to 10 nm, between 10 to 40 nm or any value in between.

In some embodiments, second distance D2 is determined based on a required optical absorption of specific wavelengths in gold structure. In some embodiments, second distance D2 is determined as to cause the sensor to generate a surface plasmon polariton at a specific wavelength. For example, when second distance D2 is 5 nm the absorbed wavelength in gold structure is 500 nm, which is typical for 5 nm In yet another example, when second distance D2 is 10 nm the absorbed wavelength in gold structure is 550 nm, which is typical for gold nanoparticles with radios of 15 nm.

In some embodiments, second distance D2 is determined based on at least one absorption wavelength of a specific analyte, wherein the absorption wavelength refers to at least one peak of the corresponding Raman spectrum of the specific analyte.

In some embodiments, first distance D1 is determined based on a required optical absorption of specific wavelengths and for sensing application, for example, guided electromagnetic wave is propagating in a transparent medium located between two extremely close metallic regions. This is used to enhance the sensing signal. In case molecule absorbs in ˜500 nm and using Raman laser of ˜528 nm then we expect to observe Raman shift at ˜1060 cm⁻¹ therefor its better to be in resonance with the molecule absorbance to increase the sensitivity.

In some embodiments, pattern 4 is a three-dimensional (3D) pattern.

In some embodiments, the thickness of the pattern is between 10 to 100 nm, for example, between 10 to 50 nm, between 10 to 20 nm, between 10 to 80 nm or any value in-between.

Reference is now made to FIGS. 10A, 10B, and 10C which are illustrations of units (upper images) and full patterns (lower images) according to some embodiments of the invention. As shown in FIGS. 10A, 10B and 10C using similar units having different first and second distance D1 and D2 may result in absorption of different wavelengths.

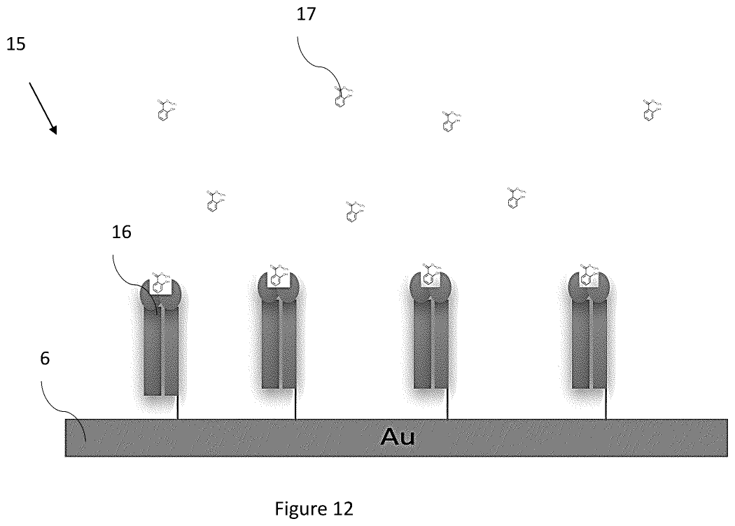

Some nonlimiting examples of pattern units are illustrated in FIG. 11. In some embodiments, a pattern may include more than one type (e.g., geometry) of units. In some embodiments, each parent may include n identical units. Alternatively, pattern 4 may include n units from a first type and m units from a second type. Wherein n and m are integers.

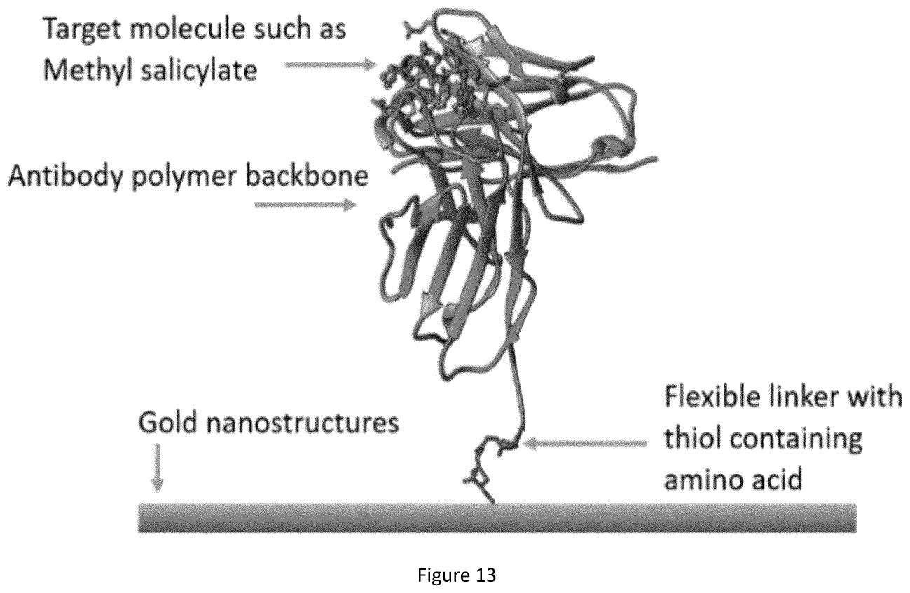

Reference is now made to FIG. 12 which is an illustration of a plurality of antibodies 16 attached to gold line 6 of pattern 4. For example, antibodies 16 covalently are attached to gold line 6 of pattern 4 via a sulfuric bond. In some embodiments, antibodies 16 are selected to be bonded to a specific analyte, for example, the methyl salicylate illustrated in FIG. 12. Some non-limiting examples for antibodies may include single-chain Fv fragments (scFv), antibody fragment-antigen binding (Fab) units and antibody single domain (sdAb). In some embodiments, binding of each antibody/antibody fragment to the surface of the gold line 6 may be performed by engineering the antibody to expose Cysteine amino acid (contain thiol group) at specific position of the antibody structure.

In some embodiments, a molecule is bound to a surface of gold pattern 4. In some embodiments, the molecule is or comprises an antibody bound to the surface of gold pattern via a covalent bond or via a non-covalent bond (e.g. by physisorption). In some embodiments, the antibody is covalently bound to a linker, wherein the linker is covalently bound to the gold surface via a thiol group. In some embodiments, the linker is as described hereinabove. In some embodiments, the molecule the selected from: a substituted or an unsubstituted mercaptoalkyl, mercaptoaryl, mercaptoalkaryl, dialkyl sulfide, diaryl sulfide or a combination thereof.

In some embodiments, there is an apparatus comprising the article of the invention in operable communication with a metallic tip, wherein upon movement of the metallic along a first direction over the outer surface of the microparticle, DC triboelectricity is generated. In some embodiments, the apparatus comprises a metallic tip located in close proximity to the outer surface of the microparticle. In some embodiments, the tip is configured to generate friction with the outer surface of the microparticle.

In some embodiments, the apparatus of the invention is described in greater detail in the Appendix attached herein.

### Uses for the Sensor

A sensor according to some embodiments of the invention may be used to detect a variety of compounds emitted in variety of industrial and agricultural field. For example, the sensor can detect analytes and odors emit in various agricultural processes, for example, by pests such as the red spider mite. In yet another example, the sensor can be used to detect emissions of undesired analytes during food production, cosmetics production and the production of pharmaceutical products. In some embodiments, the size of lines 6 in each unit 5A and 5B and the distances D1 and/or D2 may be determined based on the analyte to be detected.

### General

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed formulation, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

### Example 1

### Synthesis of Gold Microplates

Hydrogen tetrachloroaurate trihydrate (HAuCl4·3H2O), Aniline and Ethylene glycol (EG) were all purchased from Sigma-Aldrich and used without further purification.

Gold microplates were synthesized as follows. In a typical experiment 50 ml of EG is stirred and heated in an Erlenmeyer flask at 95° C. and 0.036 mmol of HAuCl4·3H2O is added to the solution in solid form. The color turns immediately to bright golden yellow indicating the dissociation of HAuCl4 and formation of tetrachloroaurate (AuCl4−) ions. 0.1 M of 0.072 mmol Aniline is then added to the solution after 20 minutes and the color changes to dark yellow in the first few seconds indicating the beginning of the oxidation and reduction of aniline and gold precursor. The magnetic stirrer is then stopped after 5 minutes and the color gradually changes to red. The reaction mixture is kept for 3 hours without any stirring. In the end of the reaction, golden sand-like particles were adhered to the surface of the flask and were easily removed by mild sonication and then is centrifuged and re-dispersed in ethanol several times.

Numerous reaction parameters, such as the ratio of the gold precursor to aniline (Auₚ:An), gold precursor concentration, temperature and time were interchanged and investigated. The different parameters are summarized in Table 1 below.

The gold micro/nano plates are then drop-casted on silicon wafers for characterization by secondary electron microscope SEM.

TABLE 1
exemplary reaction parameters tested by the inventors

| Auₚ:An | Au concentration | Temperature | | Time |
|---|---|---|---|---|
| (molar ratio) | (mg/mL) | (° C.) | | (hours) |
| 1:2 | 2.76 | 95° | C. | 3 |
| 1:2 | 2.76 | 95° | C. | 0.5 |
| 1:2 | 2.76 | 95° | C. | 1 |
| 1:2 | 2.76 | 95° | C. | 2 |
| 1:2 | 2.76 | 95° | C. | 24 |
| 1:2 | 2.76 | 95° | C. | 48 |
| 1:2 | 2.76 | 95° | C. | 168 |
| 1:2 | 2.76 | 50° | C. | 3 |
| 1:2 | 2.76 | 130° | C. | 3 |
| 1:2 | 2.76 | 25° | C. | 3 |
| 0.1:1 | 0.27 | 95° | C. | 3 |
| 0.5:1 | 1.35 | 95° | C. | 3 |
| 10:2 | 27.6 | 95° | C. | 3 |
| 10:2 | 27.6 | 95° | C. | 0.5 |
| 10:2 | 27.6 | 95° | C. | 1 |
| 10:2 | 27.6 | 95° | C. | 2 |
| 20:4 | 54.3 | 95° | C. | 3 |

### Effect of Au:Aniline Ratios:

The Au:Aniline ratio was further changed and optimized to obtain gold plates ranging from a few hundred nanometers (FIG. 1A) a to several tens of microns in edge length size (see FIG. 1B). Additionally, different shapes of 1D and 3D particles were observed. Tuning the Au:Aniline ratio to 10:0.3 resulted in long gold nanowires after conducting the reaction for five hours. Formation of 3D icosahedra nano particles was observed, by implementing Au:Aniline ratio of 10:1 after 30 minutes reaction, as shown in FIG. 1D.

### Effect of Au:Aniline Concentrations:

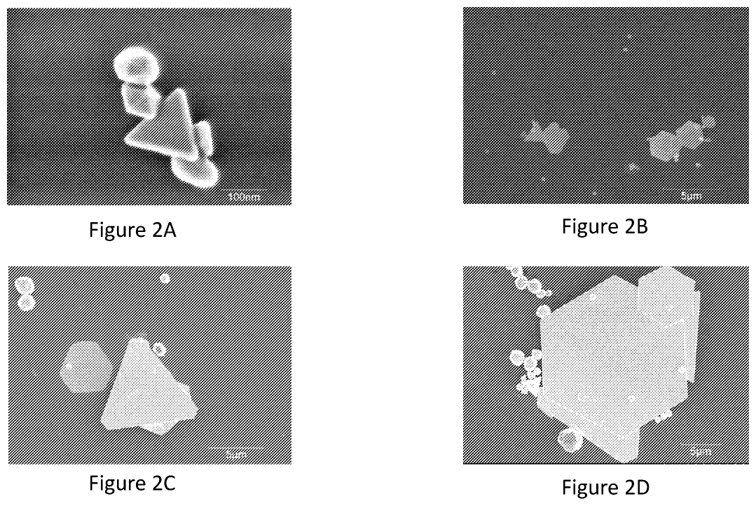

Furthermore, when the Au:Aniline ratio is shifted by a factor of 0.1, 10 and 20 the microscopy images indicates the synthesis of nano- and microplates. FIG. 2 shows the resulting plates from the ratio of 0.1:0.1. Several nanoplates of edge lengths smaller than 300 nm are obtained. As the ratio is altered to a factor of 10 and 20 the resulting plates are larger in size than the standard reaction in FIG. 2B (average size is 9 and 11 μm respectively) and gave a very high yield of microplates FIG. 2C-D.

### Effect of Reaction Time and Reaction Temperature:

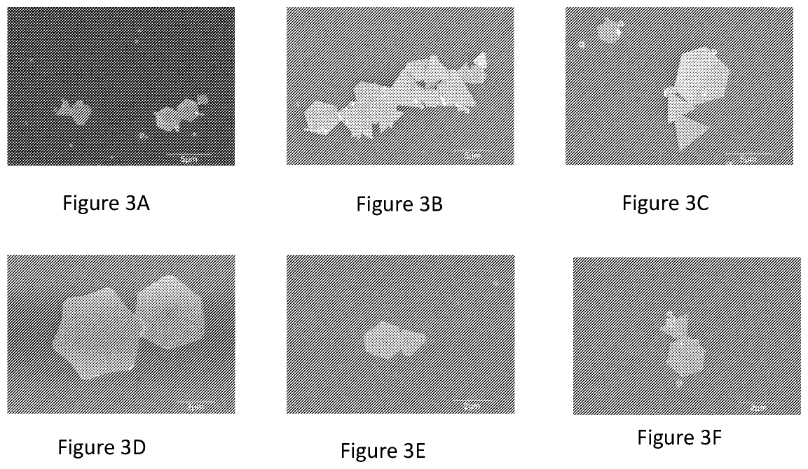

FIGS. 3A-3C show SEM images of microplates synthesized from the standard reaction as a function of time at the same temperature. In the course of one week the resulting plate average size increased by roughly a micron in length (˜3.77 μm). The synthesized plates at different reaction temperatures are indicated in FIGS. 3D-3F. The reactions were done for the same reaction time with the exception of the room temperature reaction which was done in a course of a week due to the slow speed of the reaction. Interestingly in FIG. 3D, at room temperature the resulting plates were of different shapes and yielded higher polygons. At 50° C. the resulting plates were smaller in size and thinner in thickness.

### Effect of Gold and Aniline:

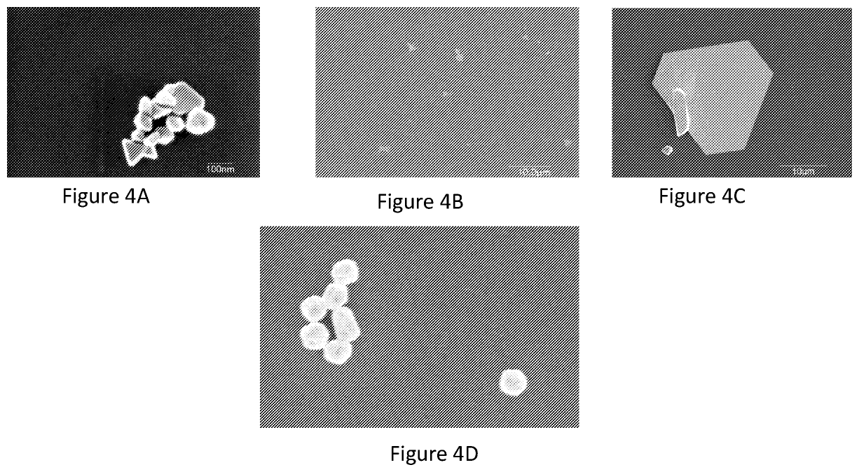

The results shown in FIGS. 4A-4C indicate the effect of increasing the gold precursor while keeping the aniline amount constant. The sizes were substantially increased when Au:Aniline ratio is 10:1 in comparison to the standard reaction (Average size ˜18 μm). Conversely the size decreased to the nanoscale when a Au:Aniline ratio of 0.1:1 is used. After tuning the aniline ratio while the gold precursor is kept constant, it can be seen in FIG. 4D that the increase of aniline promotes growth of irregular and spherical particles. So it is evident that the size of the plates is strongly influenced by the amount of precursor used.

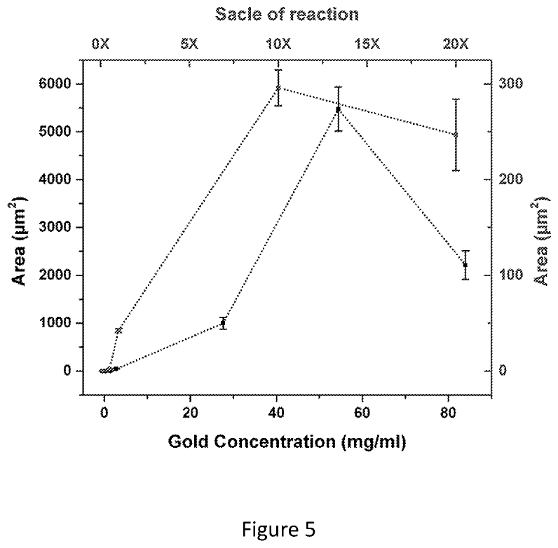

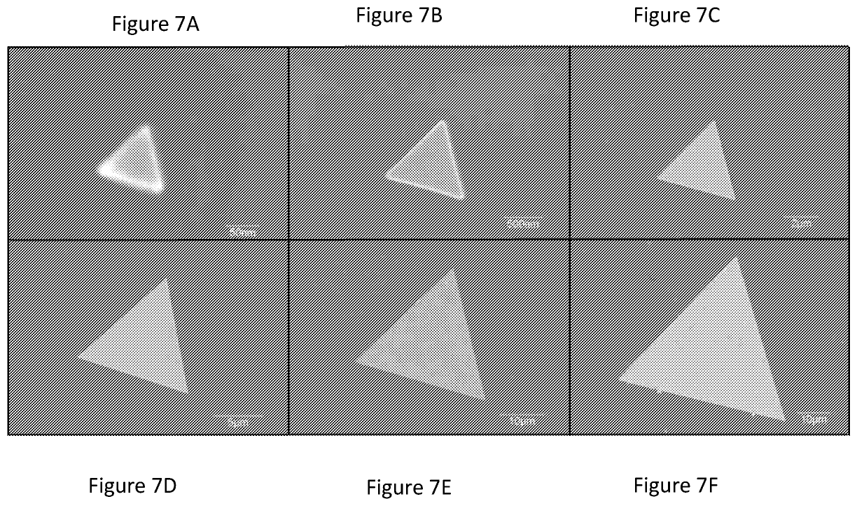

It has been postulated, that upon addition of higher or lower amounts of Gold precursor while maintaining time and temperature to be constant, plates of large areas up to square millimeter scale can be obtained (FIGS. 5 and 7). However, further addition of gold concentration beyond a certain limit yields plates of lower surface areas possibly due to the deficiency of stabilizer concentration in comparison to precursor concentration (FIG. 7). On the onset of reaction scale increase (i.e same amounts of precursor and stabilizer are used but multiplied with a factor), average area is increased but not as significantly as the gold precursor. This is due to the comparably high availability of stabilizer to synthesize multiple plates rather than focusing on the synthesis fewer but larger plates. When the reaction is scaled down, nanoplates are yielded with an average area of a few square micrometers, which is the same average area as when the gold precursor concentration is decreased. This implies that aniline plays a minor role for the synthesis of these nanoplates.

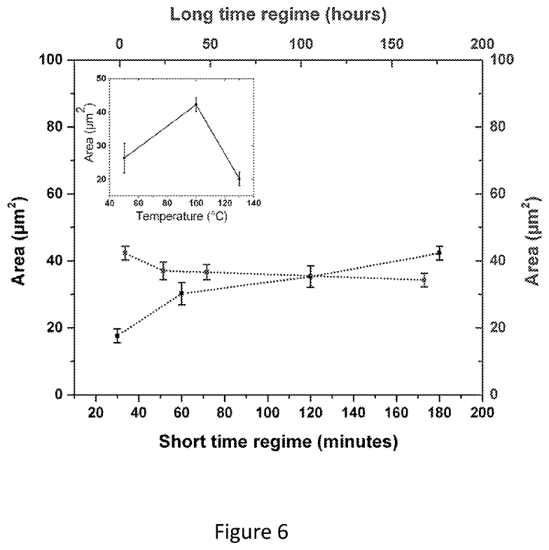

Studying the effect of temperature and time, the average area increases steadily but not strongly during the first 3 hours of the reaction and is more or less maintained throughout a week. This implies that the reaction is completed after 3 hours and no significant size increase is apparent. However, by increasing gold precursor concentration and investigation the effect of time in the first 3 hours, the average area increases more significantly and almost linearly (see FIG. 6). Increasing or decreasing the reaction temperature yields smaller sized particles due reduced reaction speed in the case of lower temperature and immediate reduction of gold precursor at elevated temperature due to the increased reducing power of Ethylene Glycol yielding plates of smaller size and several irregular particles. When the reaction is conducted at room temperature, the reaction speed is significantly slower and incomplete particles are apparent. However, if the reaction mixture is left for an extended period (e.g. a week) plates of higher polygon shapes are apparent (see FIGS. 1 and 4).

The plates synthesized have different morphologies of either a triangular, hexagonal or truncated triangular shapes. It is possible to fine tune the reaction parameters to achieve minimized or maximized sizes of the resulting plates and from analysis. The gold precursor concentration is the most effective parameter as it increases the average area by a factor of 1⁰⁴ compared to other parameters which are at least an order of magnitude smaller.

To this end, the size of the gold plates is controllable by varying the reaction parameters as illustrated in FIG. 8.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A sensor comprising:
a substrate; and
a gold pattern attached to the substrate,
wherein the gold pattern is made from a plurality of repeating units, each unit is made from at least one line having a width of between 100 to 500 nm and a length of between 1 to 50 microns, and wherein a first distance between two neighboring units is between 50 to 1000 nm,
and wherein all lines in the pattern, are originated from a monocrystalline gold microparticle, therefore, have the same crystallographic orientation with respect to the substrate.

2. The sensor of claim 1, wherein each unit comprises more than two lines or more than two segments in a line, and wherein a second distance between the two lines or the two segments is between 1 to 50 nm.

3. The sensor according to claim 2, wherein the second distance is determined based on a required optical absorption of specific wavelengths.

4. The sensor according to claim 2, wherein the second distance is determined as to cause the sensor to generate a surface plasmon polariton at a specific wavelength.

5. The sensor of claim 1, wherein the first distance is determined based on a required optical absorption of specific wavelengths.

6. The sensor of claim 1, wherein the pattern is a three-dimensional (3D) pattern.

7. The sensor of claim 1, wherein the thickness of the pattern is between 10 to 100 nm.

8. The sensor of claim 1, wherein the substrate is a dielectric substrate selected from, silicon wafer, glass, polymer, silica, and any ceramic material.

9. The sensor of claim 1, further comprising an antibody attached to the gold pattern.

10. The sensor of claim 9, wherein the antibody is covalently attached to the gold pattern via a sulfuric bond.

11. The sensor of claim 1, comprises a molecule bound to a surface of said gold pattern, the molecule is selected from: a substituted or an unsubstituted mercaptoalkyl, mercaptoaryl, mercaptoalkaryl, dialkyl sulfide, diaryl sulfide or a combination thereof.

12. A microparticle, wherein:
at least 99.999% by weight of said microparticle consist of a monocrystalline gold;
a surface area of said microparticle particle is between 0.006 mm² and 1 mm;
a thickness of said microparticle is between 10 and 100 nm.

13. The microparticle of claim 12, wherein a width dimension or a length dimension of said microparticle is between 0.5 um and 300 um.

14. The microparticle of claim 12, wherein said microparticle is a two-dimensional (2D) microparticle.

15. The microparticle of claim 12, being in a form of a uniform monolayer layer.

16. (canceled)

17. The microparticle of claim 15, wherein a surface roughness of said uniform layer is between 0.1 and 2 nm.

18. The microparticle of claim 15, wherein a permittivity of said microparticle is between 0.3 and 0.7.

19. The microparticle of claim 12, further comprises a molecule bound to a surface of said microparticle, and wherein said molecule comprises a thiol group comprising a substituted or an unsubstituted mercaptoalkyl, mercaptoaryl, mercaptoalkaryl, dialkyl sulfide, diaryl sulfide or a combination thereof.

20. (canceled)

21. The microparticle of claim 19 wherein said molecule comprises a plurality of molecules in a form of a monolayer.

22. The microparticle of claim 19, wherein said bound is via a covalent bond, or via an ionic bond.

23. (canceled)

24. (canceled)

25. (canceled)

26. A method of manufacturing a microparticle for a sensor, comprising
a. providing a reaction mixture comprising a gold salt, a reducing agent, and a solvent;
b. providing said reaction mixture to a temperature of between 25 and 150° C. for a time period of between 60 min and 5 h;
c. aging said reaction mixture for a time period of between 60 min and 7 days, thereby obtaining said microparticle; wherein:
said gold salt comprises Au(III) cation;
said reducing agent is capable of reducing said Au(III) cation to an elemental state; and wherein a concentration of said gold salt within said reaction mixture is between 0.03 and 0.3 mM,
wherein said microparticle comprises:
at least 99.999% by weight of said microparticle consist of a monocrystalline gold;
a surface area of said microparticle particle is between 0.006 mm² and 1 mm;
a thickness of said microparticle is between 10 and 100 nm.

27.-30. (canceled)
